(19) United States
(12) Patent Application Publication
(10) Pub. No.: US 2026/0226407 A1
(43) Pub. Date: Aug. 6, 2026

(54) A Mixture Of Probiotic Bacterial Strains To Improve The Digestibility Of Food Components

(71) Applicant: GIULIANI S.P.A., Milano (IT)

(72) Inventors: Giammaria GIULIANI, Montagnola (CH); Fabio RINALDI, Milano (IT); Marco GOBBETTI, Perugia (IT); Raffaella DI CAGNO, Bari (IT); Daniela PINTO, Milano (IT); Antonio MASCOLO, Milano (IT); Barbara MARZANI, Carbonara Al Ticino (IT)

(21) Appl. No.: 19/148,501

(22) PCT Filed: Jan. 17, 2024
(86) PCT No.: PCT/EP2024/051036

§ 371 Date: Jul. 16, 2025

(30) Foreign Application Priority Data
Jan. 19, 2023 (IT) 102023000000753

(51) Int. Cl. C12N 1/205; A61K 9/00; A61K 9/16; A61K 9/48; A61K 35/00; A61K 35/747; A61K 36/28; A61K 36/53; A61K 36/9068; A61K 38/47; A61K 38/48; A61K 47/44; C12R 1/225; C12R 1/25

(52) U.S. Cl. CPC C12N 1/205; A61K 9/0095; A61K 9/1617; A61K 9/1652; A61K 9/4816; A61K 9/485; A61K 9/4866; A61K 35/747; A61K 36/28; A61K 36/53; A61K 36/9068; A61K 38/47; A61K 38/482; A61K 47/44; C12Y 302/01023; C12Y 304/21026; A61K 2035/115; C12R 2001/225; C12R 2001/25

(57) ABSTRACT

The present invention relates to a nutritional composition containing selected probiotic bacteria which are lactic acid bacteria which survive in the gastrointestinal environment and are suitable to improve the digestibility of food components.

### FIELD OF THE INVENTION

The present invention relates to probiotic bacterial strains which improve the digestibility of food ingredients.

The present invention origins in the field of nutrition and of nutritional and dietetic products.

Specifically, the present invention relates to a composition containing selected probiotic bacteria which are lactic acid bacteria which survive in the gastrointestinal environment and are suitable to improve the digestibility of food components.

### BACKGROUND OF THE INVENTION

Probiotics are live microorganisms that, when administered in adequate amounts, confer a health benefit on the host.

The use of probiotic bacteria in dietary supplements or nutritional products such as yogurt, is known since long time. In the nutritional field, evidence of a health benefit is required for probiotic bacteria, at either a strain-specific or group level, depending on the nature of the benefit.

Probiotics can have different means of administration, target host species i.e. humans and animals, target populations, target sites such as gut and beyond, efficacy end points and regulatory categories. In addition, all probiotics should be safe for their intended use.

Most of the products containing probiotics available in the marked are in the form of dietary supplement or nutritional supplements for oral administration. The market of these products has grown considerably in the last ten years due to the valuable nutritional properties of certain microorganisms and their safety of use.

Typically, at present most of the probiotic formulations available in the market contains Bifidobacterium or Lactobacillus a genus of Gram-positive, aerotolerant anaerobes or microaerophilic, rod-shaped, non-spore-forming bacteria.

Strains of Bacillus sp., Pediococcus sp., and some yeasts such as Saccharomyces boulardii are also reported to be suitable probiotic candidates, as reported by Lv et al., 2014; Mingmongkolchai and Panbangred, 2018; Pereira de Paula et al., 2021.

Typically, the nutritional products containing probiotics as active ingredients are administered through the oral route with the aim of preserving eubiosis in the gastrointestinal tract or restoring the physiological conditions for the growth of the intestinal bacterial flora.

One of the main drawbacks of probiotics is the difficulty in surviving some harsh conditions found in many foods and in the human gut (Cook et al., 2012).

Accordingly, at present, there is a need of finding new uses for probiotic and dietetic products containing lactic bacteria while ensuring their resistance to the harsh conditions of the gastrointestinal environment.

A general aim of the present invention resides in providing new uses in the nutritional field for probiotics, especially for certain lactic acid bacteria (LAB).

A further aim of the present invention resides in providing a probiotic or probiotic mixtures for oral administration that improve the digestibility of food components in the gastrointestinal tract.

Another aim of the invention resides in providing dietary supplements or nutritional supplements containing probiotic bacteria selected to improve the digestibility of nutritional ingredients and to survive through the gastrointestinal tract of human beings.

### SUMMARY OF THE INVENTION

In one aspect the present invention concerns the selection of a multi-species probiotic that covers a wide range of functional features correlated with an improved digestibility of nutritional components of foodstuff. Advantageously, the probiotic bacteria of the invention are lactic acid bacteria strains (LAB).

In accordance with an aspect, the inventors firstly, have isolated from a large number of lactic acid bacteria, some strains which survive in conditions simulating the gastrointestinal environment and secondly have screened the isolated LAB for functional features linked with food digestibility enhancement. Advantageously, the strains have been isolated from natural sources such as animal niches, dairy products, fresh fruits and vegetables, and fermented vegetables (sauerkraut).

In the frame of an extensive search program, the inventors investigated the metabolic traits of lactic acid bacteria and their capability of increasing the fermentation of food and its digestibility to increase the absorption of valuable nutritional components in the gastrointestinal tract, especially in the small intestine.

In particular, the inventors investigated the metabolic traits of food grade LAB linked to their capability of increasing the digestibility health benefits, fermenting the in vitro digesta of representative food matrices (chickpeas, cheese, pomegranate, tomato and bread), and analyzing the bioavailability of derived nutritional components.

Preferably one or more of:
i) peptide hydrolysis
ii) bioavailability of phenolic compounds of fermented food matrices
iii) hydrolysis of raffinose (MRS media supplemented with raffinose) and
iv) peptidase activity
   are considered as the main metabolic traits to select the best performing potential probiotics in accordance with the invention. Accordingly:
firstly, the inventors selected Lactobacillus strains suitable for surviving in the conditions of the gastrointestinal tract among four-hundred-sixty-two Lactobacillus strains which were isolated from dairy products such as milk and cheeses, fruits and vegetables such as avocado, carrot, table olives, pineapple, tomato, fermented food products such as sauerkraut and sourdough, and animal niches such as Drosophila melanogaster, Apis mellifera and human intestine, and
secondly the strains which survived in an environment simulating the conditions of the gastrointestinal tract have been further screened for functional features linked to food digestibility enhancement.

Specifically, the inventors assessed metabolic traits such as peptidase and raffinose hydrolysis, as well as the ability to hydrolyze proteins and enhance the bioavailability of phenolic compounds in mimicked digesta of representative food matrices.

The final output has been the definition of a three-strain probiotic, which covers the widest range of assayed functional properties.

Accordingly, in a first aspect, the present invention relates to a mixture of two or more probiotic bacteria, advantageously Lactic acid bacteria, selected from the group consisting of

Lactobacillus plantarum (Lactiplantibacillus plantarum) IT1 deposited at DSMZ with accession number DSM 33940,

Lactobacillus plantarum (Lactiplantibacillus plantarum) K2 deposited at DSMZ with accession number DSM 33941,

Lactobacillus paracasei (Lacticaseibacillus paracasei) 31a deposited at DSMZ with accession number DSM 33942.

The above strains have been deposited on Jul. 15, 2021 in accordance with the Budapest Treaty at the Leibnitz-Institute DSMZ-Deutsche Sammlung von Mikroorganismen und Zellkilturen GmbH as reported in the enclosed Certificates.

The inventors found that the above selected bacterial strains improve the digestibility of nutritional components of food and foodstuff and the absorption of digestion products in the gastrointestinal tract and, advantageously, the bioavailability of micronutrients contained therein.

In the present disclosure the term bioavailability refers to the proportion or fraction of a nutrient, consumed in the diet, that is absorbed and utilized by the body of a mammal, especially of human beings.

In a further aspect, the invention relates to a Lactobacillus plantarum strain, wherein said strain is chosen from the group consisting of Lactobacillus plantarum IT1 deposited at DSMZ with accession number DSM 33940 and Lactobacillus plantarum K2 deposited at DSMZ with accession number DSM 33941 and to a Lactobacillus paracasei strain, wherein said strain is Lactobacillus paracasei 31a deposited at DSMZ with accession number DSM 33942.

Advantageously, the three selected strains of probiotic bacteria according to the invention are resistant to the gastrointestinal environment especially to gastric and intestinal fluids.

Advantageously, the three selected strains of probiotic bacteria according to the invention are also provided with food digestibility which means that they are provided at least with peptidase activity and raffinose hydrolysis and preferably also for the bioavailability of total phenolic compounds and/or release of peptides after fermentation.

Preferably, the digestibility of food or food matrices/components may be proven with food containing proteins such as cheese and bread, food containing phenolic compounds such as tomato and pomegranate and food containing raffinose and/or protein such as chickpea flour as evidenced in the detailed description of the Lactobacillus plantarum (Lactiplantibacillus plantarum) K2 deposited at DSMZ with accession number DSM 33941,

Lactobacillus paracasei (Lacticaseibacillus paracasei) 31a deposited at DSMZ with accession number DSM 33942.

The above strains have been deposited on Jul. 28, 2021 in accordance with the Budapest Treaty at the Leibnitz-Institute DSMZ-Deutsche Sammlung von Mikroorganismen und Zellkilturen GmbH as reported in the enclosed Certificates.

The inventors found that the above selected bacterial strains improve the digestibility of nutritional components of food and foodstuff and the absorption of digestion products in the gastrointestinal tract and, advantageously, the bioavailability of micronutrients contained therein.

In the present disclosure the term bioavailability refers to the proportion or fraction of a nutrient, consumed in the diet, that is absorbed and utilized by the body of a mammal, especially of human beings.

In a further aspect, the invention relates to a Lactobacillus plantarum strain, wherein said strain is chosen from the group consisting of Lactobacillus plantarum IT1 deposited at DSMZ with accession number DSM 33940 and Lactobacillus plantarum K2 deposited at DSMZ with accession number DSM 33941 and to a Lactobacillus paracasei strain, wherein said strain is Lactobacillus paracasei 31a deposited at DSMZ with accession number DSM 33942.

Advantageously, the three selected strains of probiotic bacteria according to the invention are resistant to the gastrointestinal environment especially to gastric and intestinal fluids.

Advantageously, the three selected strains of probiotic bacteria according to the invention are also provided with food digestibility which means that they are provided at least with peptidase activity and raffinose hydrolysis and preferably also for the bioavailability of total phenolic compounds and/or release of peptides after fermentation.

Preferably, the digestibility of food or food matrices/components may be proven with food containing proteins such as cheese and bread, food containing phenolic compounds such as tomato and pomegranate and food containing raffinose and/or protein such as chickpea flour as evidenced in the detailed description of the invention.

In accordance with another aspect, the inventors also developed a new strategy to select probiotic bacteria suitable to improve the digestibility of food components such as proteins, phenolic compounds as well as antinutritional compounds like raffinose.

In accordance with certain aspects, the selected strains and the mixture of this invention may be formulated as a composition for oral administration which is suitable and intended to accelerate the digestion of food, nutritional products, foodstuffs and food components.

In accordance with this aspect, a composition, especially a nutritional composition, is provided comprising the mixture of two or more the lactobacilli strains or comprising the single lactobacilli selected from Lactobacillus plantarum IT1 deposited at DSMZ with accession number DSM 33940, Lactobacillus plantarum K2 deposited at DSMZ with accession number DSM 33941, Lactobacillus paracasei 31a deposited at DSMZ with accession number DSM 33942 and mixtures thereof and a physiologically acceptable or edible carrier.

Advantageously, the composition is a nutritional composition, especially for oral administration.

In certain embodiments the composition is or is contained in a food composition, nutritional product or food supplement or dietary supplement.

The compositions containing the above referred selected lactic acid bacterial strains improve and/or accelerate the digestibility of food components and advantageously, the absorption of digested products and the assimilation of nutritional components contained therein.

A further aspect of the present invention thus relates to the use of the composition comprising the mixture of two or more of the selected lactic acid bacterial strains or the single bacterial strains taken alone, for improving the digestibility of food, food components, foodstuff or nutritional ingredients.

The composition contains the selected Lactobacilli strains/Lactic acid bacteria, which colonize the gastrointestinal tract of the individual in need of treatment.

The inventors have also discovered that the composition containing the selected Lactobacilli strains maintains eubiosis in the gastrointestinal tract.

This effect reduces the risks of colonization by pathogens and the correlated risks of developing gastrointestinal diseases.

### BRIEF DESCRIPTION OF THE FIGURES

The invention will now be described in detail and in reference to the attached Figures wherein:

FIG. 1 shows graphs illustrating the survival rate of 462 lactic acid bacteria strains under simulated gastro-intestinal conditions organized by species and annotated by isolation source, as described hereinbelow. Survival of 462 lactic acid bacteria strains under simulated gastro-intestinal conditions organized by species and annotated by isolation source.

FIG. 2 shows graphs illustrating the growth kinetics of high resistant strains incubated at 30° C. for 24 h in mMRS (modified MRS) containing raffinose (20 g/L) as the single carbon source. Color annotation depicts the isolation source of each strain.

FIG. 3 shows bar graphs illustrating the distribution of the area under the curve (AUC) computed from kinetic data. Box-plot representing strains distribution based on their capacity of growth in mMRS, determined by optical density (OD₆₂₀ values. Data were obtained in duplicate. The center line of the box represents the median, the top and bottom of the box represent the 75th and 25th percentile of the data, respectively (A). Residual raffinose concentration after 24 h of growth in mMRS (B). The 25^{th} and 75^{th} percentile corresponded to AUC of 7.30 and 22.44, while the median value was 14.04.

FIG. 4 shows bar graphs illustrating aminopeptidase type N (PepN, EC 3.4.11.11) and proline iminopeptidase (PepI, EC 3.4.11.9) activities of lactic acid bacteria strains. One unit of activity was defined as the amount of enzyme required to liberate 1 μmol of p-NA per min under the assay conditions. (A) Box-plot are also shown, representing the strains distribution based on their pepI and pepN activity. Data were obtained in duplicate. The central line of the box represents the median, the top and bottom of the box represent the 75th and 25th percentile of the data, respectively. (B) Average of peptidase activity towards leucine and proline p-nitroanilides, also showing the different source of isolation for each strain. Bars of standard deviations are shown.

FIG. 5 shows Box-plot of peptides released by lactic acid bacteria strains determined after 6 and 24 h of incubation. Data were obtained in duplicate. The central line of the box represents the median, the top and bottom of the box represent the 75^{th} and 25^{th} percentile of the data, respectively.

FIG. 6 shows Box plots of total phenolic content expressed as mg of gallic acid equivalent in 1 L of digested matrices. Data were obtained in duplicate. The central line of the box represents the median, the top and bottom of the box represent the 75^{th} and 25^{th} percentile of the data, respectively.

### DETAILED DESCRIPTION OF THE INVENTION

The present invention origins from the finding that certain strains of lactic acid bacteria belonging to the Lactobacillus species isolated from natural sources such as animal niches, dairy products, fresh fruits and vegetables, and fermented vegetables survive in the gastrointestinal environment and are suitable for digesting nutritional components of food. These specific probiotic strains have been isolated and tested for their capability to digest food and food components.

Particularly, the inventor assessed peptidase and raffinose hydrolysis metabolic traits as well as the ability to hydrolyze proteins and enhance the bioavailability of phenolic compounds in mimicked digesta of representative food matrices. The final output of the search program was the definition of three probiotic strains and in particular a combination thereof, which covers the widest range of assayed functional properties.

According to a main aspect, the invention concerns the mixture of two or more selected probiotic strains defined in claim 1 and uses thereof as defined herein.

In another aspect the present invention relates to the Lactobacillus plantarum strains chosen from the group consisting of Lactobacillus plantarum IT1 deposited at DSMZ with accession number DSM 33940 and Lactobacillus plantarum K2 deposited at DSMZ with accession number DSM 33941; and to the Lactobacillus paracasei 31a strain deposited at DSMZ with accession number DSM 33942.

Preferably the selected probiotic strains are lactic acid bacteria improving the digestibility of food components more preferably providing peptide hydrolysis, peptidase activity, raffinose hydrolysis, advantageously providing the bioavailability of phenolic compounds of fermented food matrices.

According to another aspect, the invention provides a composition comprising a Lactobacillus bacterial strain or a mixture thereof of claims 1-3.

The composition for oral administration herein disclosed may contain one or more of the following selected strains:

| Strains | Code | Source of isolation |
|---|---|---|
| Lactiplantibacillus plantarum DSM 33940 | IT1 | Fruits/vegetables |
| Lactiplantibacillus plantarum DSM 33941 | K2 | Fruits/vegetables |
| Lactiplantibacillus paracasei DSM 33942 | 31a | Fruits/vegetables |

and mixture thereof.

Preferably a mixture of the above three strains is hereby provided.

Advantageously, the mixture and the composition of this invention are intended for use in the treatment of diseases in which there is a difficulty in the digestion of food and for enhancing gastrointestinal digestion, for improving the digestibility of food, food components, foodstuff or nutritional ingredients.

In accordance with this use the composition is administered by the oral route. Accordingly, a further aspect the invention concerns with the use of a composition containing the mixture of the two or more lactobacilli of the strains defined in claim 1 and the strains according to claims 2 and 3 and an edible carrier for the digestibility of food components.

The inventors found out that the herein disclosed strains, either alone or in a mixture, after oral administration colonize the gastrointestinal tract, especially the small intestine, and exert a prolonged digestion effect of food.

The herein described selected strains of LAB find industrial application in the preparation of compositions, such as nutritional or pharmaceutical compositions, for oral administration.

In the composition the strain and their combination is mixed with a carrier.

The term “carrier” refers to a vehicle, excipient, diluents, or adjuvant with which the therapeutic or active strains is administered. Any carrier and/or excipient suitable for the form of preparation desired for administration is contemplated for use with the strains disclosed herein.

The carrier may take a wide variety of forms depending on the form of preparation desired for administration, e.g. oral administration.

In certain embodiments, the strain of the present invention can be combined as an active ingredient in intimate admixture with a suitable physiological or edible carrier and/or excipient according to conventional compounding techniques in the nutritional field.

One embodiment provides a food composition or a nutritional supplement, also called as food supplement, which comprises one or more of said strain(s) or the above disclosed composition.

The food composition or the nutritional supplement may be also referred as a food product. A nutritional or food supplement is intended to provide one or more nutrients, prebiotic or probiotics, typically manufactured in a pre-dosed form, that may otherwise not be consumed in sufficient quantities.

The food composition or nutritional supplement may contain as auxiliary agents, fillers, amino acids and/or proteins, fatty acids, carbohydrates, vitamins, minerals, botanical extracts obtained from plants or cell cultures, enzymes, functional ingredients such as Citicoline, Epigallocatechin gallate, Beta-Hydroxy-Beta-Methyl Butyrate (HMB), pharmaceutical agents, preservatives, flavours, aroma or the like. Food/feed compositions include canine foods in any form.

Examples of nutritional supplements include tablets in all forms such as coated, uncoated, orodispersible, fast dissolving, hard and soft capsules, ready to disperse powder packaged in sachet or stick pack, bottles paired with a chamber caps containing lyophilized probiotics for extemporaneous reconstitution/preparation, oil suspension (powder in oil).

Example of food and functional food include snacks, treats, fermented milk-based products, chewing products, yoghurt, kefir, and the like milk products.

Fermented milk products, which also known as cultured dairy foods, cultured dairy products, or cultured milk products, are dairy food products that have been fermented with lactic acid bacteria.

In one embodiment the composition is a nutritional product.

In one embodiment the composition or supplement contains also other probiotic strains including various lactic acid bacteria, Bifidobacteria, and/or Saccharomyces. Especially when the strain or culture described here is used in fermented dairy products it should be compatible with fermenting microbes and possible further probiotic strains.

One embodiment provides the composition or the supplement, wherein the bacteria are lyophilized, tyndallized, in the form of a suspension or spray dried.

In order to maintain their biological activity the strains, lyophilized, dried, or liquid culture, can be introduced aseptically to heat-treated food/feed composition.

One embodiment provides the composition or the supplement, wherein the bacteria are lyophilized or spray dried.

The strains of LAB disclosed herein may be used in methods wherein the strain, or a culture containing said strain, is to be administered to a subject in need thereof.

The strain or the culture may be in the form of any of the food products described herein.

The effective amount as used herein refers to a daily dose which is sufficient for providing and maintaining a positive effect in the subject after either a single treatment or multiple administrations.

In another particular embodiment, the invention also refers to compositions of the strains of this invention in a lyophilized, freeze-dried or dried form, which can be obtained by any conventional method known in the art.

In another particular embodiment, the probiotic strain or the mixture thereof is in the form of non-viable cells.

In another embodiment, the feed or nutritional product comprising a probiotic strain of the invention is in a lyophilized, freeze-dried or dried form, which can be obtained by any conventional method known in the art.

In another embodiment of the feed or nutritional product, the probiotic strain or the mixture thereof is in the form of non-viable cells.

The compositions may be prepared by any of the methods well-known in the field of nutritional science.

In some embodiments, in the compositions of nutritional products of the present invention the strains are generally formulated in dosage units and prepared by any of the methods well-known in the art of pharmacy.

When used in combination with one or more other active ingredients, the strain of the present invention and the other active ingredient may be used in lower doses than when each is used singly.

In certain embodiments, the composition may contain an amount of the above identified bacterial strains from 0.00001% to 10%, from 0.0001 to 3%, from 0.01 to 2% weight with respect to the total weight of the composition.

In some embodiments, the amount of the probiotics herein disclosed is in the range from 0.001% by weight to about 60% by weight of the formulation/composition.

The composition may be conveniently presented in unit dosage form.

For example, the dosage unit may contain from 100.000.000 to 20.000.000.000 ufc/dosage unit of living bacterial strain, or from 0.00001 to 1000 mg of each strain per dosage unit for daily administration.

Additional features of the invention are described in the following detailed description that includes some tests carried out to isolate and select the Lactobacilli showing enhancement in the food digestibility.

Advantageously, the inventors first selected LAB strains able to survive under simulated GI conditions among four-hundred-sixty-two (462) LAB strains previously isolated from dairy products (milk and cheeses), fruits and vegetables (e.g. avocado, carrot, table olives, pineapple, tomato), fermented food products (sauerkraut and sourdough), and animals niches (Drosophila melanogaster, Apis mellifera and human intestine). Selected resistant strains have been further screened for functional features linked to food digestibility enhancement as described hereinbelow.

### Culture Collection

Food-grade LAB species (with a current Qualified Presumption of Safety status as judged by the EFSA scientific Panels), mainly Lactobacillus spp., Pediococcus spp. and Leuconostoc spp, and belonging to Micro4Food Culture Collection of the Libera Universitá di Bolzano were used (Table 1).

TABLE 1
Lactic acid bacteria belonging to Micro4Food Culture
Collection selected for GIT survival challenge

| | | | Number |
|---|---|---|---|
| | | | of |
| Source | Source Subtype | Specie | strains |
| Animal | Apis melifera | Lactiplantibacillus | 1 |
| | intestine | plantarum | |
| | Drosophila | Levilactobacillus brevis | 2 |
| | melanogaster | L. plantarum | 1 |
| | intestine | | |
| | Feces | L. plantarum | 1 |
| Dairy | Cheese | L. plantarum | 2 |
| | Milk | L. brevis | 4 |
| | | Lactobacillus curvatus | 7 |
| | | Lactobacillus gasseri | 1 |
| | | Lactobacillus helveticus | 2 |
| | | Lactobacillusparabuchneri | 3 |
| | | Lacticaseibacillus | 27 |
| | | paracasei | |
| | | L. plantarum | 1 |
| | | Pediococcus pentosaceus | 1 |
| Flour | Spelt flour | L. curvatus | 1 |
| | Oat flour | L. plantarum | 1 |
| Fruits and | Apple | L. brevis | 1 |
| Vegetables | | L. paracasei | 3 |
| | Avocado | L. plantarum | 1 |
| | Carrot | L. plantarum | 2 |
| | Cherry | L. plantarum | 1 |
| | Fennel | L. plantarum | 1 |
| | | L. plantarum | 1 |
| | Grape | L. plantarum | 2 |
| | Kiwi | L. plantarum | 5 |
| | Olives | Lactobacillus pentosus | 2 |
| | | L. plantarum | 1 |
| | Papaya | L. plantarum | 1 |
| | Pineapple | L. plantarum | 3 |
| | | Furfurilactobacillus | 3 |
| | | rossiae | |
| | Prune | L. plantarum | 3 |
| | Sauerkraut | L. paracasei | 1 |
| | | L. plantarum | 2 |
| | | Leuconostoc holzapfelii | 7 |
| | | Leuconostoc | 4 |
| | | mesenteroides subsp. | |
| | | jonggajibkimchii | |
| | | Leuconostoc suionicum | 1 |
| | | Pediococcus parvulus | 9 |
| | Tomato | L. plantarum | 7 |
| | | P. pentosaceus | 1 |
| Other | Other | L. plantarum | 2 |
| | | F. rossiae | 1 |
| | | Weissella confusa | 1 |
| | Textile bag | Pediococcus acidilactici | 1 |
| Sourdough | Sourdough S1 | L. plantarum | 3 |
| | | Lacticaseibacillus | 6 |
| | | rhamnosus | |
| | Sourdough S43 | L. paracasei | 3 |
| | | Lactobacillus plajomi | 1 |
| | | L. plantarum | 20 |
| | | Lacticaseibacillus | 2 |
| | | rhamnosus | |
| | Sourdough S44 | L. curvatus | 10 |
| | | Limosilactobacillus | 5 |
| | | fermentum | |
| | | L. pentosus | 8 |
| | | L. plantarum | 23 |
| | | Leuconostoc citreum | 36 |
| | | Leuc. holzapfelii | 1 |
| | | Leuconostoc | 1 |
| | | mesenteroides | |
| | | Leuconostoc | 7 |
| | | pseudomesenteroides | |
| | Sourdough S69 | L. plantarum | 52 |
| | | L. rhamnosus | 3 |
| | | F. rossiae | 1 |
| | Sourdough S88 | L. pentosus | 5 |
| | | L. plantarum | 143 |
| | | L. rhamnosus | 4 |
| | Tritordeum dough | P. pentosaceus | 3 |
| | | L. curvatus | 2 |
| | | L. plantarum | 1 |
| Grand Total | | | 462 |

### First Screening: Resistance to Simulated Gastric and Intestinal Fluids Under In Vitro Conditions

The aim of the first screening was the selection of strains based on their capability to resist under the simulated GI conditions. Initially, 462 strains were subjected to simulated gastric and intestinal fluids, as described by (Fernández, 2003). Stationary-phase-grown cells were harvested (7500 rpm for 10 min), washed with physiologic solution, and suspended (cell density of ca. 9 log CFU/ml) in 50 ml of simulated gastric juice, which contains NaCl (125 mM/l), KCl (7 mM/l), NaHCO₃ (45 mM/l), and pepsin (3 g/l) (Sigma-Aldrich CO., St. Louis, MO, USA) (Zárate et al., 2000). After that, final pH was adjusted to 2.0 and, effect of gastric digestion was also determined by suspending cells in reconstituted skimmed milk (RSM) (11% solids, w/v) with simulated gastric juice at adjusted pH of ca. 3.0. The suspension was incubated at 37° C. under anaerobic conditions and stirring to simulate peristalsis and the effect of the food matrix during gastric transit (Zárate et al., 2000). After 3 h of gastric digestion, cells were harvested and suspended in simulated intestinal fluid, which contains pancreatin (0.1%, w/v) and Oxgall bile salt (0.15%, w/v) (Sigma-Aldrich Co.) at pH 8.0. The suspension was incubated at 37° C. under aerobic and stirring conditions for 3 more h. Cell density was monitored at different timepoints (0, 3, and 6 h) according to De Angelis et al. (2006).

### Second Screening: Characterization of Target Functional Features.

The aim of this step was to evaluate the 47 selected high-resistant strains for the following functional features to subsequently formulate a combination which includes three strains which together outperform in wide range of such activities.

### Raffinose Hydrolysis

Raffinose hydrolysis was also evaluated through the determination of the growth kinetic parameters of the selected 47 strains in modified MRS broth, which contained raffinose (20 g/L) as the only carbon source. Bacterial growth was monitored by measurement of optical density (OD) at 620 nm over 24 h. Growth kinetic parameters such as area under the curve (AUC), growth rate value (μ, h⁻¹) and maximum height of the curve (A) were determined. After 24 h of incubation, residual raffinose was determined by using a Raffinose/D-Glucose Assay kit according to the manufacturer's instructions (Megazyme Raffinose/D-Glucose Assay Kit—Megazyme International, Ireland). Bacterial growth was monitored by measurement of optical density (OD) at 620 nm after 24 h.

### Peptidase Activity Towards Leu-p-Na and Pro-p-Na Synthetic Substrates

Peptidase activities of 47 selected resistant strains were assayed using synthetic substrates, which contained leucine or proline bond to a p-nitrophenyl group. Cells were harvested by centrifugation (10.000 rpm for 10 min at 4° C.), washed with sterile 50 mM potassium phosphate buffer (pH 7.0), re-suspended in the same buffer at a 620 nm absorbance (A₆₂₀) of 2.5, which corresponded to a cell density of ca. 9.0 log CFU/ml. The reaction mix was added (80 μl of 50 mM phosphate buffer pH 7; 20 μl 20 mM of Leu-p-Na and Pro-p-Na substrate in methanol; and 2 μl NaN₃ 5%) to an eppendorf containing 100 μl of cell suspension. Two blanks were prepared for each assay; one with PBS in replacement of the corresponding substrate and second with the addition of PBS instead of cell suspension. Samples were incubated under stirring conditions (150 rpm) at 30° C. for 1 and 24 h for Leu-p-Na and Pro-p-Na, respectively. The reaction was stopped by adding 500 μl of 10% acetic acid. Then, samples were centrifuged (10,000 rpm for 10 min) and the absorbance was read at 410 nm, resetting against substrate blank.

### Simulation of Digestion of Five Targeted Food Matrices

Partially digested targeted food matrices were prepared as described by Brodkorb et al. (2019), with slightly modifications. In particular, cheese and bread (proteins), tomato and pomegranate (phenolic compounds), and chickpea flour (raffinose and proteins) were chosen as most representative matrices containing the main food components for which the digestibility should be demonstrated. Briefly, the digestion procedure involved three phases: preparation, digestion, and sample treatment. Food matrices were exposed to two sequential digestive phases: oral and gastric phase. Food was first diluted (1:1, w/w) with simulated salivary fluid (SSF), together with CaCl₂ (1.5 mM), salivary amylase (75 U/ml), and incubated for two min while mixing (37° C., pH 7.0) to simulate the oral phase. The oral bolus was then diluted (1:1, w/w) with simulated gastric fluid (SGF), together with CaCl₂) (0.15 mM), and gastric enzymes (pepsin and gastric lipase, 60 U/ml) and incubated for two h while mixing (37° C., pH 3.0). The partially digested matrices were then incubated for 24 h with single resistant strains to GI conditions preselected in the first screening. In order to have an indirect response of the capability of LAB strains to digest the above food matrices, indicators such as total peptides and phenolic compounds were evaluated after 6 and 24 h of incubation.

### Determination of Total Peptides

Peptide concentration was determined by o-phthaldialdehyde OPA method as described by Sah et al. (2014) with some modifications. Total peptides were expressed as mg of tryptone equivalents per 1 ml of sample.

### Determination of Total Free Phenolic Compounds

Folin-Ciocalteu method was used for the determination of total free phenolic compounds according to Singleton and Rossi (1965) as described by Di Cagno et al. (2017). Analyses were carried out on the supernatant after centrifuging the samples at 10.000 g for 10 min. Total phenolic compounds were expressed as mg gallic acid equivalent (GAE) per 1 Litre of sample. A standard curve prepared using tryptone (0 to 500 mg/L) was used as the reference.

### Results

### Selection of High-Resistant Strains

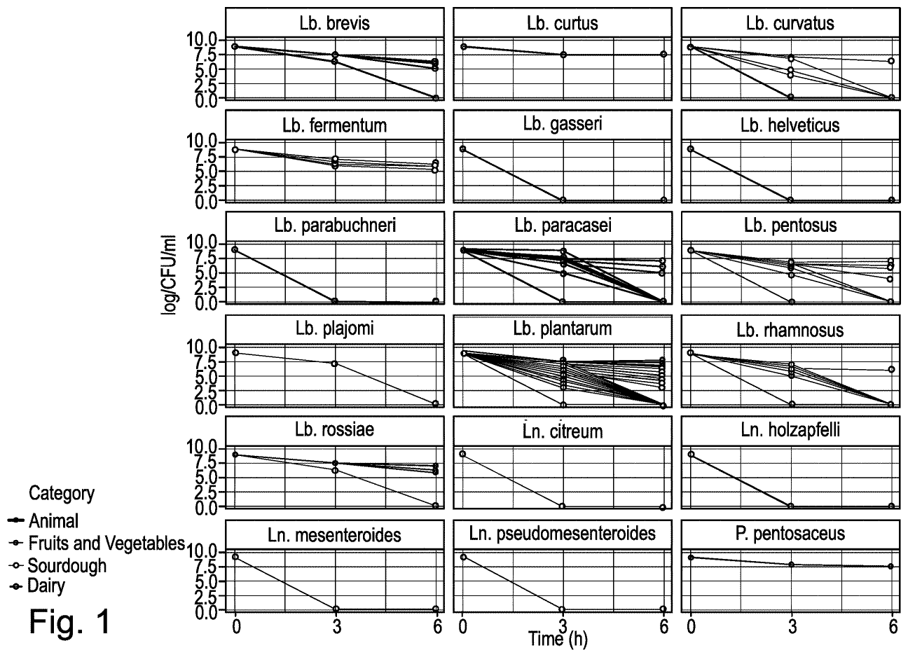

After 3 h of incubation under simulated gastric juice, 117 strains showed a decrease of growth lower than ca. 3 log cycle compared to the initial cell density (ca. 9 log CFU/ml) (FIG. 1). Sixty-seven strains showed a reduction of cell density lower than 3 log CFU/ml after 6 h of incubation. Forty-seven strains were selected as high resistant to simulated GIT conditions because the decrease of cell density after 6 h of incubation was less than 2 log CFU/ml. These strains belong to L. brevis (1), L. paracasei (1), L. plantarum (35), L. rhamnosus (1), F. rossiae (2), P. acidilactici, (1) P. pentosaceus (5), and W. confusa (1). Furthermore, 20 strains showed an intermediate resistance to GI conditions because of the decrease of cell density after 6 h of incubation, which was between 2 and 3 log CFU/ml. Intermediate resistant strains belonged to the following species: L. brevis (2), L. curvatus (1), L. fermentum (2), L. paracasei (1), L. pentosus (5), L. plantarum (6), L. rhamnosus (1), F. rossiae (1) and Leuconostoc holzapfelii (1). In total, 67 out of 462 strains showed a g resistance to the harsh environment imposed by GI conditions. For the following screening, 47 out of 67 outperforming candidates, high resistant strains were selected as illustrated in the following Table 2.

TABLE 2
Resistant and Non-resistant strains under simulated gastro-
intestinal conditions. Resistant strains are categorized
as High Resistant (total reduction of CFU/ml lower than
2 log CFU/ml) and Intermediate Resistant (total reduction
of CFU/ml between 3 and 2 log CFU/ml).

| | High | Intermediate | Not | |
|---|---|---|---|---|
| | Resistant | Resistant | resistant | Total |
| L. brevis | 1 | 2 | 4 | 7 |
| L. curvatus | 0 | 1 | 19 | 20 |
| L. fermentum | 0 | 2 | 3 | 5 |
| L. gasseri | 0 | 0 | 1 | 1 |
| L. helveticus | 0 | 0 | 2 | 2 |
| L. parabuchneri | 0 | 0 | 3 | 3 |
| L. paracasei | 1 | 1 | 32 | 34 |
| L. pentosus | 0 | 5 | 10 | 15 |
| L. plajomi | 0 | 0 | 1 | 1 |
| L. plantarum | 35 | 6 | 240 | 281 |
| L. rhamnosus | 1 | 1 | 13 | 15 |
| F. rossiae | 2 | 1 | 2 | 5 |
| Leuc. citreum | 0 | 0 | 36 | 36 |
| Leuc. holzapfelii | 0 | 1 | 7 | 8 |
| Leuc. mesenteroides | 0 | 0 | 1 | 1 |
| Leuc. mesenteroides | 0 | 0 | 4 | 4 |
| subsp. jonggajibkimchii | | | | |
| Leuc. | 0 | 0 | 7 | 7 |
| pseudomesenteroides | | | | |
| Leuc. suionicum | 0 | 0 | 1 | 1 |
| P. acidilactici | 1 | 0 | 0 | 1 |
| P. parvulus | 0 | 0 | 9 | 9 |
| P. pentosaceus | 5 | 0 | 0 | 5 |
| W. confusa | 1 | 0 | 0 | 1 |
| Grand Total | 47 | 20 | 395 | 462 |

Resistance to GI transit is a prerequisite to define a strain as probiotic. Results showed that more than 85% of the studied strains didn't show an high capability to withstand such harsh conditions, since their viability decreased dramatically when subjected to simulated GI incubation. Only 47 strains maintained a viable cell population above ca. 7 CFU/mL after an in vitro GI transit performed, turning them as potential probiotic candidates (Lemos Junior et al., 2020).

### Second Part: Functional Features Screening

### Raffinose Hydrolysis

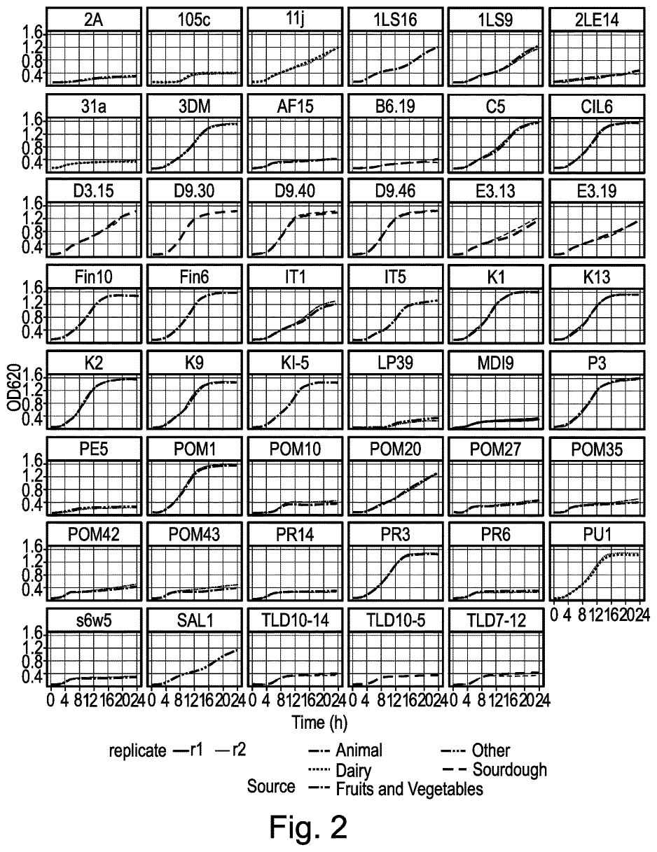

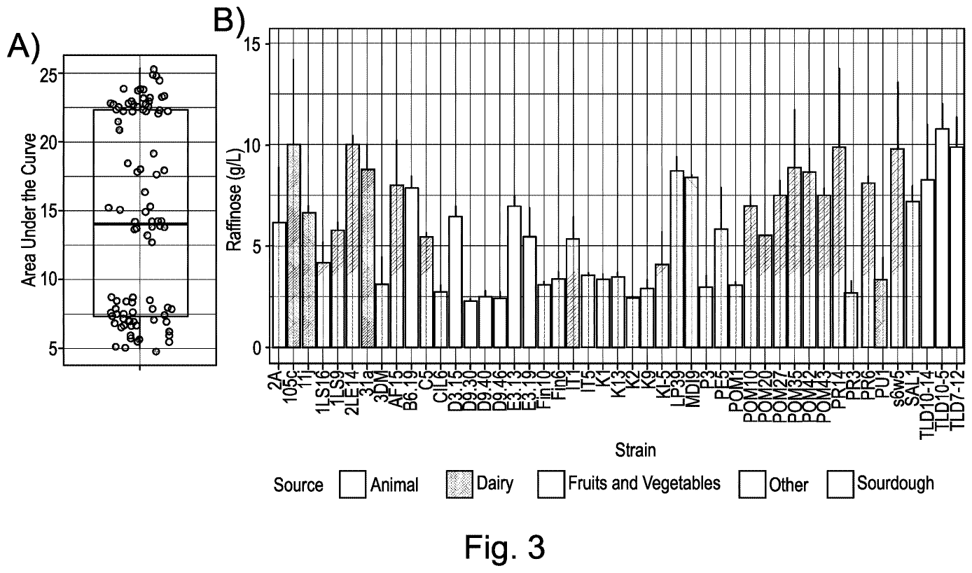

Raffinose family oligosaccharides (RFOs) in food are considered anti-nutritional factors. RFOs are not degraded in the upper GI tract due to the absence of the α-galactosidase, an intracellular enzyme that degrades raffinose to galactose and sucrose as final product of hydrolysis. The non-degradation of raffinose can cause GI symptoms that are dose dependent (Teixeira, McNeill, and Gänzle, 2012). Many LAB, due to the action of α-galactosidase activity, have been used to reduce/eliminate RFOs in food products (Montemurro et al., 2019). Hence, the selection of LAB probiotic candidates in this study has been also based on their capability to degrade antinutritional sugars and prevent digestive disorders. Therefore, the evaluation of best-performing strains for raffinose hydrolysis was carried out considering 3 growth parameters (AUC, u, and A) from the growth kinetic curves on a synthetic media containing raffinose as the only carbon source (FIG. 2), which was subsequently confirmed by determining the concentration of residual raffinose in mMRS medium after 24 h of incubation (FIG. 3).

As illustrated in FIG. 3, the AUC ranged from 4.96 to 24.88, while μ and A from 0.01 to 0.18 and from 0.16 to 5.48 OD₆₂₀, respectively. Based on AUC and μ parameters, L. plantarum POM1 and 1LS16, isolated from fruits and vegetables, showed the best performance of growth kinetics on mMRS. Regarding the raffinose hydrolysis after 24 h, results showed a variation from 2.3 up 10.7 g/L. The best strain able to hydrolyze raffinose is L. plantarum D9.30, isolated from sourdough (FIG. 3).

The capability of hydrolysis was strain dependent.

### Peptidase Activity Towards Leu-p-Na and Pro-p-Na Synthetic Substrates

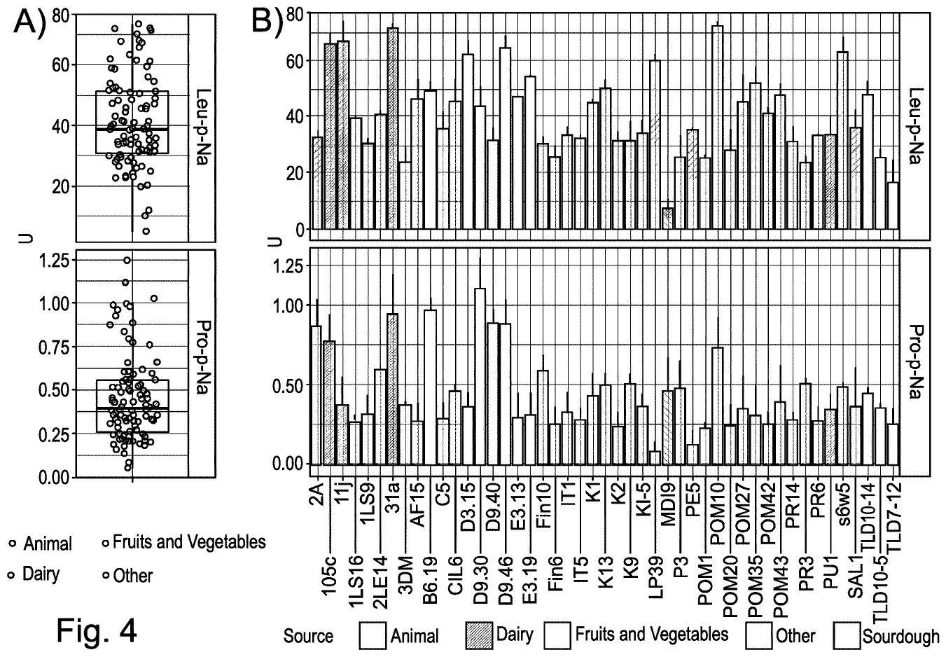

Peptidase is used to describe the capability of LAB to hydrolyse peptides and breaking them down into low molecular weight peptides or amino acids. The role of peptidases to improve the digestibility of protein based-food matrices during digestion was recently investigated (Brown et al., 2017, Di Cagno et al., 2002). Two synthetic substrates (Leu-p-Na and Pro-p-Na) were considered. Peptidase activity distribution across all strains is shown in FIG. 4A, while the average of peptidase activity per strain towards leucine and proline p-nitroanilides is shown in FIG. 4B. All Lactobacillus strains showed general aminopeptidase (EC 3.4.11.4) (PepN) activity specific for Leu-p-nitroanilides (Leu-p-NA), which ranged from 7.55 to 73 units (U) (median value, 37.1 U) (FIG. 4A and FIG. 4B). P. pentosaceus POM10, L. paracasei 31a and L. paracasei 11j showed the highest activities (73.0±0.78, 71.3±1.45, and 66.6±6.89 U, respectively). Compared to PepN activity, these strains showed a lower proline iminopeptidase (PepI) activity (median value, 0.37 U) (FIG. 4A and FIG. 4B). Strains showing the highest PepI activity were L. plantarum D9.30 and B6.19, and L. paracasei 31a (1.12±0.19, 0.98±0.07, and 0.95±0.24, respectively). As expected, the highest activity on both Leu-p-Na and Pro-p-Na was found for strains isolated from animal sources, which correlates with the fact that proteolytic activity of LAB focusing on the hydrolysis of peptides into aminoacids is well known especially during the cheese ripening (Gobbetti, Smacchi, and Corsetti, 1996).

### Total Concentration of Peptides

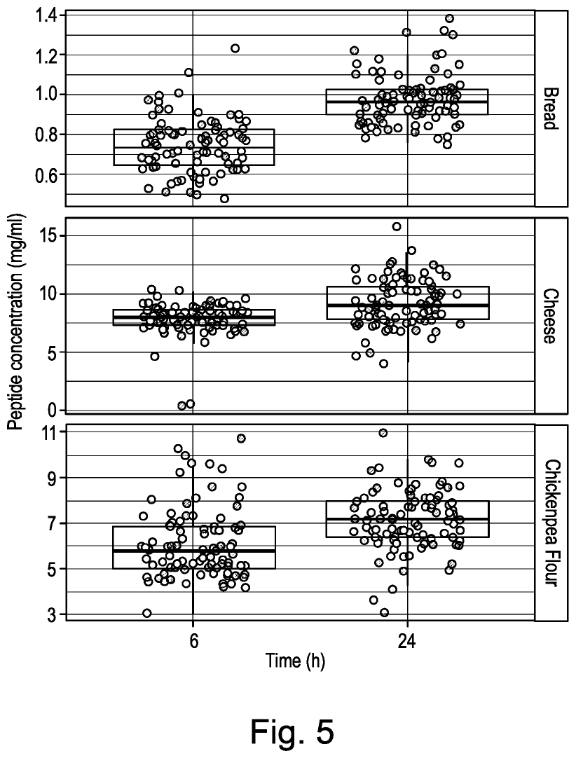

Free peptides released after 6 and 24 h of incubation with partially digested matrices (cheese, bread, chickpea flour) were measured as illustrated in FIG. 5.

As expected, total concentration of peptides was different based on the incubation time (6 and 24 h) (p<0.05, test t), and based on the digested food matrices. The highest production of peptides was found after 24 h of incubation (FIG. 5). Peptide concentration in partially digested bread ranged from 1.01±0.05 (L. plantarum PR14) to 2.4±0.17 mg/ml (L. plantarum IT1) after 6 h of incubation (median value, 1.51 mg/ml), and from 1.60±0.00 (L. plantarum D9.40) to 2.66±0.23 mg/ml (F. curtus 105c) after 24 h of incubation (median value, 1.97 mg/ml). In partially digested cheese, the concentration ranged from 0 (F. rossiae 2LE14) to 8.01±0.83 mg/ml (L. plantarum P3) after 6 h of incubation (median value, 6.23 mg/ml), and from 3.12±1.27 (L. plantarum KI-5) to 10.21±0.87 mg/ml (L. plantarum POM43) after 24 h of incubation (median value, 7.62 mg/ml). Finally, in partially digested chickpea flour the concentration ranged from 2.32±1.46 (L. pentosus E3.19) to 7.69±0.27 mg/ml (L. brevis MDI9) after 6 h of incubation (median value, 4.11 mg/ml), and from 1.61±0.39 (P. pentosaceus POM10) to 7.90±1.81 mg/ml (L. plantarum P3), after 24 h of incubation (median value, 5.46 mg/ml).

GI digestion of dietary proteins is a crucial step both to supply the organism of essential and non-essential amino acids and for the release of medium- and short-size peptides with potential biological activity. The digestibility of protein sources has been defined as the proportion of food protein derived from aminoacids, which is effectively digested and absorbed, thus becoming available adequately for the synthesis of body protein (Rizzello et al., 2012).

### Bioavailability of Total Phenolic Compounds

Phenolic compounds are secondary plant metabolites whose structure include one or more aromatic rings substituted by one or more hydroxyl groups. The interest on phenolic compounds in food is related to their health benefits including their antimicrobial features (Filannino et al., 2015). The absorption and metabolism of polyphenols in the digestive tract governs their biological properties. Only those released from the food matrix in the small and large intestine are digested. Polyphenols occur in foods mainly as esters, glycosides and polymers which cannot be absorbed in these native forms and thus require hydrolysis by digestive system enzymes or intestinal microflora (Wojtunik-Kulesza et al., 2020).

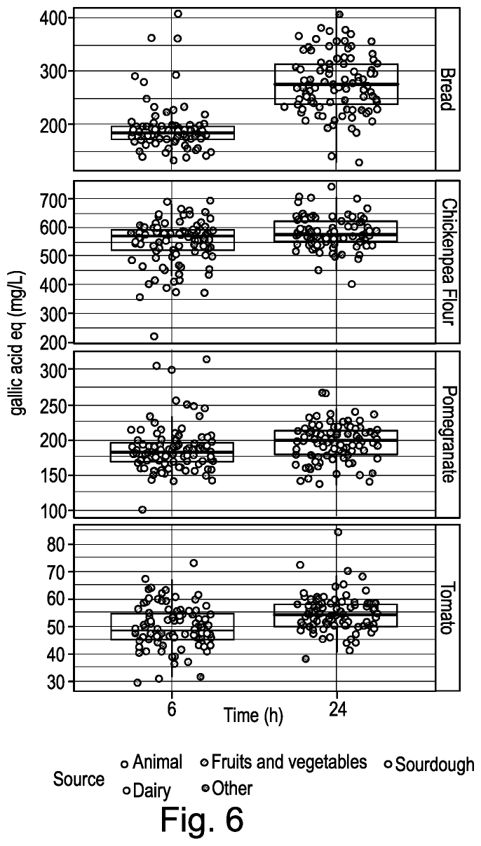

The bioavailability of total phenolic compounds after 6 and 24 h incubation with partially digested matrices (bread, chickpea flour, pomegranate and tomato) is shown in FIG. 6. Total phenolic content was significantly dependent on digested matrices. From 6 to 24 h of incubation the highest level of phenolic compounds was found as evidenced in FIG. 6. After 24 h the incubation with digested matrices, the highest concentration of total phenolic compounds was found. In particular, the highest value was found for L. paracasei 31a (703.51±1.18 mg/ml), followed by L. plantarum 3DM (356.51±70.44 mg/m), L. brevis MDI9 (232.11±50.55 mg/ml), and L. plantarum AF15 (68.95±21.07 mg/ml). The median value was different based on the digested matrice: bread (median value, 274.72 mg/ml), chickpea flour (median value, 579.70 mg/ml), pomegranate (median value, 199.54 mg/ml), and tomato (median value, 54.55 mg/ml).

Enzymes as β-glucosidase, decarboxylase, and reductase harboured by lactic acid bacteria are able to metabolize phenolic acids into the corresponding reduced derivatives, with higher biological (e.g., antioxidant and anti-inflammatory) activities than the precursors it has been showed (Tlais et al., 2020). In this study, lactobacilli isolated from fermented foods having high content of phenolic compounds show high capacity to metabolize these compounds. Moreover, the capability to metabolize these phenolic compounds is strain- or species dependent.

### Selection of Most Promising Probiotic Candidates

Briefly, starting from 462 strains (406+56) a double screening step was carried out. Based on the survival under the simulated GI conditions 47 strains (“high resistant” strains) were selected and then characterized for their peptidase activity and relative total concentration of peptides, raffinose hydrolysis, and bioavailability of total phenolic compounds and release of peptides after fermentation. Partially digested matrices were used for the determination of total peptides and phenolic compounds after 6 and 24 hours of incubation with the selected strains. The selection of the best performing strains during the second screening was done using a scoring approach. Therefore, a strain was considered positive (score 1) if the value for a given assay parameter is higher than the 3°quartile of total values for such assay, otherwise it was scored as 0. Then, the proportion of positive (%) scores for a given assay was determined by diving the total cumulative score for an assay category divided by the number of parameters for that assay (Eq. 1). For instance, Table 3 shows the scoring for three strains in OPA assay taking into account peptide concentration on different food (chickpeas, cheese, bread) matrixes after 24 h fermentation and the determination of positive proportion.

$$Assay Score = \frac{\sum positive score parameter}{total number parameter in the assay}$$

TABLE 3
Example of calculation using score method developed considering
the total peptide concentration determined by OPA method.

| | OPA | Bread OPA | Cheese OPA Chickpea |
|---|---|---|---|
| Strain | 24 h | 24 h | 24 h |
| Strain 1 | 1 | 0 | 0 |
| Strain 2 | 0 | 1 | 1 |
| Strain 3 | 0 | 0 | 0 |

According to the scores, the best candidates for each assay were chosen (Table 4). In the last step of selection, only species that are already recognized as officially probiotic were considered. For instance, F. rossiae was excluded from the combinations because at the moment this species has some regulation limitations to be defined probiotic.

TABLE 4
Scoring table of the most performing strains. Score 1 indicates that the value of a parameter for a given
assay falls in the 25% of the highest values (3^{rd} quartile of the data distribution), while score 0 indicates
that the value is below this threshold. Colored strains were chosen for the formulation of probiotics.
Yellow, best performing strains on peptidase activity assay; blue, best performing strains on OPA assay
and Folin-Ciocolteau assay; red, strains having the highest capacity to hydrolize raffinose.

| | | | | Peptide | | | | | | |
|---|---|---|---|---|---|---|---|---|---|---|
| | | Peptide | Peptide | conc. | | Total | Total | Total | Total | |
| | | conc. | conc. | Chickpea | Strain | phenol | phenol | phenol | phenol | Strain |
| | | Bread | Cheese | Flour | OPA | Bread | ChickFlour | Pomegrabate | Tomato | Folin |
| strain | source | 24 h | 24 h | 24 h | Score | 24 h | 24 h | 24 h | 24 h | Score |
| K2 | Fruits and | 0 | 0 | 0 | 0% | 0 | 1 | 0 | 0 | 25% |
| | Vegetables | | | | | | | | | |
| P3 | Fruits and | 0 | 1 | 1 | 67% | 1 | 0 | 0 | 0 | 25% |
| | Vegetables | | | | | | | | | |
| D9.46 | Sourdough | 0 | 1 | 0 | 33% | 1 | 1 | 0 | 0 | 50% |
| K13 | Fruits and | 0 | 0 | 0 | 0% | 1 | 0 | 0 | 0 | 25% |
| | Vegetables | | | | | | | | | |
| 1LS16 | Fruits and | 0 | 0 | 1 | 33% | 1 | 1 | 1 | 0 | 75% |
| | Vegetables | | | | | | | | | |
| E3.13 | Sourdough | 0 | 0 | 0 | 0% | 0 | 1 | 1 | 1 | 75% |
| 1T1 | Fruits and | 1 | 0 | 1 | 67% | 1 | 0 | 0 | 1 | 50% |
| | Vegetables | | | | | | | | | |
| 105c | Dairy | 1 | 0 | 0 | 33% | 1 | 0 | 1 | 0 | 50% |
| 31a | Dairy | 1 | 1 | 0 | 67% | 0 | 1 | 0 | 0 | 25% |
| POM10 | Fruits and | 1 | 0 | 0 | 33% | 0 | 0 | 0 | 0 | 0% |
| | Vegetables | | | | | | | | | |
| B6.19 | Sourdough | 0 | 0 | 0 | 0% | 0 | 0 | 0 | 0 | 0% |
| 2LE14 | Fruits and | 0 | 0 | 1 | 33% | 1 | 1 | 1 | 1 | 100% |
| | Vegetables | | | | | | | | | |

| | | | | Conc. | | | | |
|---|---|---|---|---|---|---|---|---|
| | | Raffinose | | raffinose | | | | |
| | Raffinose | Growth | Raffinose | after 24 h | | | | |
| | Area Under | Rate | Maximum | fermentation | Raffinose | | | Peptidase |
| strain | the Curve | (h-1) | Absorbance | (g/L) | Score | Leu-p-na | Pro-p-na | Score |
| K2 | 1 | 1 | 1 | 1 | 100% | 0 | 0 | 0% |
| P3 | 1 | 1 | 1 | 1 | 100% | 0 | 0 | 0% |
| D9.46 | 1 | 1 | 0 | 1 | 75% | 1 | 1 | 100% |
| K13 | 1 | 1 | 0 | 0 | 50% | 1 | 0 | 50% |
| 1LS16 | 0 | 0 | 1 | 0 | 25% | 0 | 0 | 0% |
| E3.13 | 0 | 0 | 1 | 0 | 25% | 0 | 0 | 0% |
| 1T1 | 0 | 0 | 1 | 0 | 25% | 0 | 0 | 0% |
| 105c | 0 | 0 | 0 | 0 | 0% | 1 | 1 | 100% |
| 31a | 0 | 0 | 0 | 0 | 0% | 1 | 1 | 100% |
| POM10 | 0 | 0 | 0 | 0 | 0% | 1 | 1 | 100% |
| B6.19 | 0 | 0 | 0 | 0 | 0% | 1 | 1 | 100% |
| 2LE14 | 0 | 0 | 0 | 0 | 0% | 0 | 1 | 50% |

Based on the selection, four combinations of LAB strains showing the best performance in all considered analyses were proposed (Table 5). In particular, five strains belonging to two species showed the best performance according our scoring approach (Table 4).

TABLE 5
Overview of four most promising probiotic combinations.
Probiotic combinations recommended

| | | | |
|---|---|---|---|
| Combination 1 | L. plantarum K2 | L. plantarum | L. plantarum IT1 |
| | | D9.46 | |
| Combination 2 | L. plantarum P3 | L. plantarum | L. plantarum IT1 |
| | | D9.46 | |
| Combination 3 | L. plantarum K2 | L. paracasei 31a | L. plantarum IT1 |
| Combination 4 | L. plantarum P3 | L. paracasei 31a | L. plantarum IT1 |

Based on the results, only 47 strains can be considered as potential probiotics considering the capability to survive GI tract conditions.

Furthermore, results from the second screening suggested that four combinations including five strains, three of them were selected as preferred according to the invention, to be suitable probiotics able to improve the digestibility of targeted food matrices.

The Combination 3 according to the invention containing a mixture of Lactobacillus plantarum IT1 deposited at DSMZ with accession number DSM 33940, Lactobacillus plantarum K2 deposited at DSMZ with accession number DSM 33941, Lactobacillus paracasei 31a deposited at DSMZ with accession number DSM 33942, is preferred.

Certain embodiments of the invention are further disclosed by way of the following examples.

### Example 1

Hard capsule

| Ingredients | quantity/unit. | |
|---|---|---|
| Lacto3 Giuliani | 210 | mg |
| Tolerase L | 25.5 | mg |
| Tolerase G | 6 | mg |
| Artichoke dry extract 2.5% | 10 | mg |

| | |
|---|---|
| Syloid 244FP | if necessary |

Vegan Capsule size 0 Low Water Activity: 1 empty capsule

Lacto3 Giuliani means a mixture of:
Lyophilized of Lactiplantibacillus plantarum K2—DSM 33941 (viable cells: 1*10 exp 9 cfu/dose),
Lacticaseibacillus paracasei 31a—DSM 33942 (viable cells: 1*10 exp 9 cfu/dose),
Lactiplantibacillus plantarum IT1—DSM 33940 (viable cells: 1*10 exp 9 cfu/dose);
Tolerase L means: Maltodextrin, Lactase (beta galattosidase),
Tolerase G means: Prolyl oligopeptidase, Maltodextrin,
Artichoke dry extract 2.5% Caffeoilquininic acids, expressed as chlorogenic acid: Artichock (Cynara scolymus L.) leaf dry extract, Maltodextrin.
Syloid 244FP: Silica
Vegan Capsule size 0 Low Water Activity: Hypromellose

### Example 2

### Sachet—Water Dispersible (Extemporaneous) Powder

| Ingredients | quantity/unit | |
|---|---|---|
| Lacto3 PLUS Giuliani | 300 | mg |
| Chamomile dry extract | 20 | mg |
| Ginger dry extract | 20 | mg |
| Maltodextrin | 800 | mg |
| Strawberry flavour | 32.5 | mg |
| Citric acid anhydrous | 8 | mg |
| Steviol glycosides (E 960) from Stevia | 4 | mg |
| Syloid 244FP | 4 | mg |

Lacto3 PLUS Giuliani means a mixture of
Lyophilized of Lactiplantibacillus plantarum K2—DSM 33941 (viable cells: 4*10 exp 9 cfu/dose),
Lacticaseibacillus paracasei 31a—DSM 33942 (viable cells: 4*10 exp 9 cfu/dose),
Lactiplantibacillus plantarum IT1—DSM 33940 (viable cells: 4*10 exp 9 cfu/dose),
Chamomile dry extract: Matricaria Chamomilla flower, dry extract, maltodextrin
Ginger dry extract: Zingiber officinale rhizome dry extract, maltodextrin
Syloid 244FP: Silica.

### Example 3

### Stick Pack—Orodispersible Powder

| Ingredients | quantity/unit. | |
|---|---|---|
| Lacto3 PLUS Giuliani | 300 | mg |
| Lemon balm dry extract | 40 | mg |
| Maltodextrin | 100 | mg |
| Strawberry flavour | 15 | mg |
| Citric acid anhydrous | 7 | mg |
| Steviol glycosides (E 960) from Stevia | 3.5 | mg |
| Syloid 244FP | 4 | mg |

Lacto3 PLUS Giuliani: Lyophilized of Lactiplantibacillus plantarum K2—DSM 33941 (viable cells: 4*10 exp 9 cfu/dose), Lacticaseibacillus paracasei 31a—DSM 33942 (viable cells: 4*10 exp 9 cfu/dose), Lactiplantibacillus plantarum IT1—DSM 33940 (viable cells: 4*10 exp 9 cfu/dose).

Lemon balm dry extract: Melissa officinalis L. leaf dry extract, Maltodextrin

Syloid 244FP: Silica

### Example 4

### Stick Pack—Oil Suspension 5 mL

| Ingredients | quantity/unit. |
|---|---|
| Lacto3 M-PLUS Giuliani | 250 million/strain for |
| | each dose (6 drops) |
| Sunflower oil high oleic | 5 mL |

Administering 6 drops a day in a single intake.

Shake before use; 6 drops provide 250*10 exp 6 viable cells of each strain.

Lacto3 M-PLUS means: Microencapsulated strains of Lactiplantibacillus plantarum K2—DSM 33941, Lacticaseibacillus paracasei 31a—DSM 33942, Lactiplantibacillus plantarum IT1—DSM 33940.

Relating to Deposited

| | | |
|---|---|---|
| 0-1 | Form PCT/RO/134 | |
| | Indications Relating to Deposited | |
| | Microorganism(s) or Other Biological | |
| | Material (PCT Rule 13bis) | |
| 0-1-1 | Prepared Using | ePCT |
| | | Version 4.12 MT/FOP 20240212/1.1 |
| 0-2 | International Application No. | PCT/EP2024/051036 |
| 0-3 | Applicant's or agent's file reference | P022493WO-01 |
| 1 | The indications made below relate to | |
| | the deposited microorganism(s) or | |
| | other biological material referred to in | |
| | the description on: | |
| 1-1 | page | 3 |
| 1-2 | line | 32 |
| 1-3 | Identification of deposit | |
| 1-3-1 | Name of depositary institution | DSMZ Leibniz Institute DSMZ-German Collection of Microor- |
| | | ganisms and Cell Cultures |
| 1-3-2 | Address of depositary institution | Leibniz Institute DSMZ-German Collection of Microorganisms |
| | | and Cell Cultures |
| | | Inhoffenstr. 7B |
| | | D-38124 Braunschweig |
| | | Germany |
| 1-3-3 | Date of deposit | 15 Jul. 2021 (15 Jul. 2021) |
| 1-3-4 | Accession Number | DSMZ 33940 |
| 1-4 | Additional Indications | |
| 1-5 | Designated States for Which | All designations |
| | Indications are Made | |
| 1-6 | Separate Furnishing of Indications | |
| | These indications will be submitted to the | |
| | International Bureau later | |
| 2 | The indications made below relate to | |
| | the deposited microorganism(s) or | |
| | other biological material referred to in | |
| | the description on: | |
| 2-1 | page | 4 |
| 2-2 | line | 2 |
| 2-3 | Identification of deposit | |
| 2-3-1 | Name of depositary institution | DSMZ Leibniz Institute DSMZ-German Collection of Microor- |
| | | ganisms and Cell Cultures |
| 2-3-2 | Address of depositary institution | Leibniz Institute DSMZ-German Collection of Microorganisms |
| | | and Cell Cultures |
| | | Inhoffenstr. 7B |
| | | D-38124 Braunschweig |
| | | Germany |
| 2-3-3 | Date of deposit | 15 Jul. 2021 (15 Jul. 2021) |
| 2-3-4 | Accession Number | DSMZ 33941 |
| 2-4 | Additional Indications | |
| 2-5 | Designated States for Which | All designations |
| | Indications are Made | |
| 2-6 | Separate Furnishing of Indications | |
| | These indications will be submitted to the | |
| | International Bureau later | |
| 3 | The indications made below relate to | |
| | the deposited microorganism(s) or | |
| | other biological material referred to in | |
| | the description on: | |
| 3-1 | page | 4 |
| 3-2 | line | 4 |
| 3-3 | Identification of deposit | |
| 3-3-1 | Name of depositary institution | DSMZ Leibniz Institute DSMZ-German Collection of Microor- |
| | | ganisms and Cell Cultures |
| 3-3-2 | Address of depositary institution | Leibniz Institute DSMZ-German Collection of Microorganisms |
| | | and Cell Cultures |
| | | Inhoffenstr. 7B |
| | | D-38124 Braunschweig |
| | | Germany |
| 3-3-3 | Date of deposit | 15 July 2021 (15.07.2021) |
| 3-3-4 | Accession Number | DSMZ 33942 |
| 3-4 | Additional Indications | |
| 3-5 | Designated States for Which | All designations |
| | Indications are Made | |
| 3-6 | Separate Furnishing of Indications | |
| | These indications will be submitted to the | |
| | International Bureau later | |

FOR RECEIVING OFFICE USE ONLY

| | | |
|---|---|---|
| 0-4 | This form was received with the | YES |
| | international application: | |
| | (yes or no) | |
| 0-4-1 | Authorized officer | Rossi, Cinzia |

FOR INTERNATIONAL BUREAU USE ONLY

| | | |
|---|---|---|
| 0-5 | This form was received by the interna- | 23 Feb. 2024 (23 Feb. 2024) |
| | tional Bureau on: | |
| 0-5-1 | Authorized officer | |

1-9. (canceled)

10. A mixture of the probiotic bacteria selected from the group consisting of Lactobacillus plantarum IT1 deposited at DSMZ with accession number DSM 33940, Lactobacillus plantarum K2 deposited at DSMZ with accession number DSM 33941 and Lactobacillus paracasei 31a deposited at DSMZ with accession number DSM 33942.

11. A Lactobacillus plantarum strain, wherein said strain is chosen from the group consisting of Lactobacillus plantarum IT1 deposited at DSMZ with accession number DSM 33940 and Lactobacillus plantarum K2 deposited at DSMZ with accession number DSM 33941.

12. A composition comprising the mixture according to claim 10 and a physiologically acceptable carrier.

13. The composition according to claim 12, wherein said composition is for oral administration.

14. The mixture according to claim 10, wherein the bacteria are in vivo, tyndallized, in the form of a suspension or lyophilized.

15. The strains according to claim 11, wherein the bacteria are in vivo, tyndallized, in the form of a suspension or lyophilized.

16. A nutritional supplement or dietary supplement comprising the composition according to claim 12 and an ingredient selected from the group consisting of auxiliary agents, fillers, amino acids, proteins, fatty acids, carbohydrates, vitamins, minerals, botanical extracts obtained from plants or cell cultures, enzymes, Citicoline, Epigallocatechin gallate, Beta-Hydroxy-Beta-Methyl Butyrate (HMB), pharmaceutical agents, preservatives, flavours and aroma.

17. A nutritional supplement or dietary supplement comprising the composition according to claim 13 and an ingredient selected from the group consisting of auxiliary agents, fillers, amino acids, proteins, fatty acids, carbohydrates, vitamins, minerals, botanical extracts obtained from plants or cell cultures, enzymes, Citicoline, Epigallocatechin gallate, Beta-Hydroxy-Beta-Methyl Butyrate (HMB), pharmaceutical agents, preservatives, flavours and aroma.
